(19) United States
(12) Patent Application Publication
(10) Pub. No.: US 2024/0335482 A1
(43) Pub. Date: Oct. 10, 2024

(54) BACTERIAL TREATMENT FOR ATOPIC DERMATITIS

(71) Applicants: Ryan WALTERS, Atlanta, GA (US); Eric KOEPP, Williston, VT (US)

(72) Inventors: Ryan WALTERS, Atlanta, GA (US); Eric KOEPP, Williston, VT (US)

(21) Appl. No.: 18/627,124

(22) Filed: Apr. 4, 2024

Related U.S. Application Data
(60) Provisional application No. 63/494,282, filed on Apr. 5, 2023.

(51) Int. Cl. A61K 35/74; A61K 9/12; A61K 9/19; A61K 36/9064

(52) U.S. Cl. CPC A61K 35/74; A61K 9/12; A61K 9/19; A61K 36/9064

(57) ABSTRACT

A technique for treating atopic dermatitis is disclosed, comprising the steps of preparing a culture of R. mucosa and applying it to the affected skin to create a colony of R. mucosa in situ. The R. mucosa colony competes with S. aureus, a common exacerbator of atopic dermatitis, and is hypothesized to have direct action on atopic dermatitis, not mediated by S. aureus.

### FIELD OF THE INVENTION

The subject matter of this application pertains to methods and compositions for treating skin disorders, particularly dermatitis. Even more particularly, the subject matter of this application pertains to treatments for eczema (atopic dermatitis). The subject matter of this application further pertains to a novel method to prepare and apply a bacterial-laden compound to a patient's skin. Even more particularly, the subject matter of this application pertains to novel methods and compositions for using bacteria to treat atopic dermatitis.

### BACKGROUND OF THE INVENTION

Dermatitis is a general term used for several skin irritations and rashes. Dermatitis can be caused by genetic factors, immune system disorders, or more banal factors like allergies or irritation. Most people are afflicted by dermatitis at some point in their life.

Eczema, or atopic dermatitis (AD), is a chronic skin condition that causes inflammation, redness, and irritation. It affects approximately 18 million people in the United States or approximately 1 out of every 20. AD usually begins in childhood, and flare-ups can persist throughout a person's lifetime. There is no cure for AD.

Staphylococcus aureus colonizes the skin of most patients with AD if not all. S. Aureus colonization is associated with a deficiency of antimicrobial peptides, decreased filaggrin and filaggrin degradation products, overexpressed Th2/Th17 cytokines, microbial dysbiosis, and altered lipid profiles. S. aureus colonization on AD-affected skin causes skin barrier dysfunction through superantigens, enzymes, and other proteins.

As if that isn't enough, the colonization of S. aureus also worsens AD. Skin colonization of S. aureus, particularly methicillin-resistant S. aureus (MRSA), is a significant challenge in managing AD. Attempts to control the proliferation of S. aureus include the use of topical and systemic antibiotics and extended soaks in dilute bleach solutions. However, the indiscriminate use of antibiotics can increase the occurrence of resistant bacteria, so appropriate steps must be taken to prevent the treatment from making the situation worse.

One way of combating the colonization of S. aureus is to manipulate the skin microbiome. Early research has suggested that Roseomonas mucosa (“R. mucosa”) reduces the S. aureus load, presumably reducing the bacterial-mediated exacerbation of AD. However, some parts of the effect are likely due to simple competition between bacterial species. R. mucosa may also have a more direct effect on AD, possibly through the induction of a tumor necrosis factor receptor type II-mediated pathway.

There is a need for new treatments for AD, and although adding R. mucosa to the skin microbiome is promising, there are problems associated with providing such a culture. For one, few consumers may be willing to learn enough microbiology techniques to grow a pure culture of R. mucosa at home and apply it to their skin so that it won't further irritate them. For another, bacteria don't tend to survive in a lotion or other carrier for a commercially appropriate amount of time. Indeed, lotion is generally not a nurturing environment for bacteria, and most bacteria added during manufacture are dead within 30 days.

A most desired R. mucosa-mediated topical treatment would allow a consumer to apply an amount of R. mucosa with other non-bacterial products used to treat AD and soothe irritated skin. One method that may come to mind is to mix R. mucosa into a lotion or cream. However, such a formulation would have a limited shelf life as the bacteria would die without access to a food source and oxygen.

### SUMMARY OF THE INVENTION

This application concerns methods and compounds for delivering R. mucosa to the skin and helping to re-establish an R. mucosa-laden skin microbiome.

This disclosure aims to provide a simple method of preparing and delivering R. mucosa to the skin by combining it with an appropriate topical delivery medium.

The subject matter of this application satisfies this objective. In particular, the subject matter of this application discloses several methods of preparing and applying R. mucosa to the skin. The treatment system may also comprise other bacteria or bacteria-derived compounds, medicines, probiotics, prebiotics, and other suitable skin preparations.

A treatment for AD involves preparing a solution containing R. mucosa and applying it to and around AD-affected skin.

Said R. mucosa may be in any appropriate form that is stable at room temperature. In the most highly preferred embodiment, the R. mucosa is lyophilized, as known in the art and packaged into sachets of the bacteria. In a highly preferred embodiment, the sachets further comprise an herb or spice. In a most highly preferred embodiment, this herb or spice is ground cardamon.

Testing has shown that adding ground cardamon at about 0.1% of the mass of the lyophilized R. mucosa is advantageous. Amounts of ground cardamon up to 10% of the mass of R. mucosa have been tested without harmful effects.

In a highly preferred embodiment, a sachet of lyophilized R. mucosa and ground cardamon is sealed and labeled with identifying information such as lot number, packaging date, and a best-by date when at least 50% of the lyophilized bacteria are estimated to be viable.

Later, the sachet's contents are rehydrated, applied to the affected area, and left to air dry.

Other features and advantages of the present invention will be apparent from the following more detailed description of the preferred embodiment, taken in conjunction with the accompanying drawings, which illustrate, by way of example, the principles of the invention.

### BRIEF DESCRIPTION OF DRAWINGS

FIG. 1 illustrates the steps to practice a preferred embodiment of the invention.

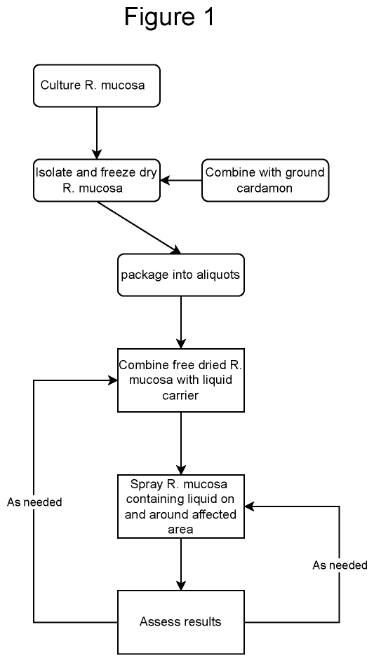

The same reference numbers will be used throughout the drawings to represent the same parts wherever possible.

### DETAILED DESCRIPTION OF THE INVENTION

The following description illustrates embodiments of the application's subject matter. It is not intended to limit the scope of any claims. Those familiar with the art will recognize that other embodiments of the disclosed method and compositions are possible. One should consider all such alternative embodiments within the application's disclosure.

Certain word choices are made for simplicity and to aid the reader's understanding by placing the subject matter of this application in context. These word choices should not be interpreted to narrow the scope of the claims, which should be given their broadest possible meaning in the light of the disclosure. For example, as used in this application: “AD” means atopic dermatitis as well as any other dermatitis; “R. Mucosa” means Roseomonas mucosa; and “lyophilized” means lyophilized or sporulated.

While the exemplary embodiments illustrated in the figures and described herein are presently preferred, these embodiments are offered by way of example only. Accordingly, the present application is not limited to a particular embodiment but extends to various modifications that fall within the scope of the appended claims. The order or sequence of any processes or method steps may be varied or re-sequenced according to alternative embodiments.

It is important to note that the construction and arrangement of the various exemplary embodiments are illustrative only. Although only a few embodiments have been described in detail in this disclosure, those skilled in the art who review this disclosure will readily appreciate that many modifications are possible (e.g., variations in sizes, dimensions, structures, shapes, and proportions of the various elements, values of parameters, mounting arrangements, use of materials, colors, orientations, etc.) without materially departing from the novel teachings and advantages of the subject matter recited in the claims. For example, elements shown as integrally formed may be constructed of multiple parts or components, the position of elements may be reversed or otherwise varied, and the nature or number of discrete elements or positions may be altered or varied. Accordingly, all such modifications are intended to be included within the scope of the present application. The order or sequence of any process or method steps may be varied or re-sequenced according to alternative embodiments. In the claims, any means-plus-function clause is intended to cover the structures described herein as performing the recited function and not only structural equivalents but also equivalent structures. Other substitutions, modifications, changes, and omissions may be made in the design, operating conditions, and arrangement of the exemplary embodiments without departing from the scope of the present application.

A treatment for AD involves preparing a solution containing R. mucosa and applying it to and around AD-affected skin.

Suitably sized cultures of R. mucosa are grown by known methods, such as adding a sample to a bioreactor containing an appropriate medium, agitating the media to maintain proper aeration, removing effluent, and collecting the bacteria.

The isolated bacteria are prepared for lyophilization (freeze drying), which may be as simple as freezing the bacteria and applying a vacuum to remove water or may include steps such as treating the bacteria before freezing with bovine serum albumin or mannitol to help maintain the shape of the bacteria as water is removed, and additional rounds of vacuum drying and temperature adjustment.

Following lyophilization, the R. mucosa is packaged in an appropriate material and sealed to maintain viability.

At this point, a dried ground spice of herb may be added to support the bacteria after rehydration, help maintain viability, or provide a pleasing scent. Our studies have indicated that as little as .1% of ground cardamom is sufficient to accomplish these goals.

We estimate between 2 and 5 grams of R. mucosa is sufficient to create a shelf-stable product that can be rehydrated and yield a useful number of viable bacteria.

For use, the sachet of R. mucosa is opened, and the contents are mixed with a suitable liquid carrier. In a most highly preferred embodiment, the carrier is distilled water, but tap or bottled water could be used. The water could have additional nutrients added, such as bovine serum albumin or a hexose. Further, in the most highly preferred embodiments, the carrier and sachet contents are combined within the body of a spray bottle.

The amount of carrier should be sufficient to hydrate the lyophilized R. mucosa. For most highly preferred embodiments, this amount is not so significant as to require more than a reasonable amount of time to spray nearly all the solution onto the afflicted area using a standard handheld atomizer and not so little that the resulting solution is too thick for easy application. In our testing, approximately 5 ml of carrier is suitable for rehydrating 2 g of lyophilized bacteria.

After adding the freeze-dried bacteria to the carrier, the solution may be left for several minutes or up to several hours to allow the bacteria to rehydrate. The time allotted for rehydration may be less than the time required to achieve complete hydration of the bacteria. Fifteen minutes of rest and gentle agitation are preferred, although the solution could rehydrate for up to 48 hours without harmful effects.

Once the bacteria are adequately hydrated, one directs the spray bottle toward an affected area and applies the solution on and around the area. Most often, the contents are sprayed until the bottle is empty.

Once spraying is stopped, the affected area is left to dry uncovered to reduce the loss of bacteria from the skin due to brushing a surface such as clothing and to increase the chances of a significant culture of R. mucosa being established in situ.

Several days or weeks after application, the severity of the treated AD may be assessed, and additional applications may be made as needed.

While the exemplary embodiments illustrated in the figures and described herein are presently preferred, it should be understood that these embodiments are offered by example only. Accordingly, the present application is not limited to a particular embodiment but extends to various modifications that fall within the scope of the appended claims. The order or sequence of any processes or method steps may be varied or re-sequenced according to alternative embodiments.

It is important to note that the construction and arrangement of the various exemplary embodiments are illustrative only. Although only a few embodiments have been described in detail in this disclosure, those skilled in the art who review this disclosure will readily appreciate that many modifications are possible (e.g., variations in sizes, dimensions, structures, shapes, and proportions of the various elements, values of parameters, mounting arrangements, use of materials, colors, orientations, etc.) without materially departing from the novel teachings and advantages of the subject matter recited in the claims. For example, elements shown as integrally formed may be constructed of multiple parts or elements, the position of elements may be reversed or otherwise varied, and the nature or number of discrete elements or positions may be altered or varied. Accordingly, all such modifications are intended to be included within the scope of the present application. The order or sequence of any process or method steps may be varied or re-sequenced according to alternative embodiments. In the claims, any means-plus-function clause is intended to cover the structures described herein as performing the recited function and not only structural equivalents but also equivalent structures. Other substitutions, modifications, changes, and omissions may be made in the design, operating conditions, and arrangement of the exemplary embodiments without departing from the scope of the present application.

1. A method of treating atopic dermatitis comprising the steps of:
providing a culture of R. mucosa,
providing said culture with conditions conducive to bacterial growth,
preparing the bacterial for freeze drying,
freeze-drying the bacteria,
packaging the previously freeze-dried bacteria with approximately .1% ground cardamon by weight,
rehydrating said lyophilized bacteria and ground cardamon with an amount of water,
spraying the bacteria solution on and around the affected skin.

2. A method of applying a bacterial solution to skin comprising the steps of:
hydrating an amount of lyophilized bacteria in an amount of water,
placing the solution into a spray bottle; and
spraying the solution on a region of skin.

3. A method of preparing and packaging lyophilized bacteria for consumer use comprising the steps of:
Providing a sample of bacteria,
Growing said sample of a bacteria to a desired concentration using standard practices,
Prepare said bacterial for lyophilization,
Freeze-drying the bacteria,
Placing an aliquot of the bacteria in a foil package; and
Sealing said foil package.
